(19) United States
(12) Patent Application Publication
(10) Pub. No.: US 2022/0362174 A1
(43) Pub. Date: Nov. 17, 2022

(54) Use of R-enantiomer Beta2-agonists for prevent and treatment of pulmonary inflammation and inflammatory remodeling for reduced adverse effects

(71) Applicant: WEN TAN, Foshan City, Guangdong (CN)

(72) Inventor: WEN TAN, Foshan City, Guangdong (CN)

(21) Appl. No.: 17/607,868

(22) PCT Filed: May 5, 2020
(86) PCT No.: PCT/US20/31539

§ 371 Date: Oct. 30, 2021

Related U.S. Application Data
(60) Provisional application No. 62/844,398, filed on May 7, 2019.

(30) Foreign Application Priority Data
Sep. 7, 2019 (US) PCT/US2019/050120

(51) Int. Cl. A61K 31/137; A61K 31/27; A61K 31/167; A61K 31/4704; A61K 31/44; A61K 31/538; A61K 31/138; A61K 31/55; A61K 31/439; A61K 31/5386; A61K 31/40; A61K 31/573; A61P 11/00

(52) U.S. Cl. CPC A61K 31/137; A61K 31/27; A61K 31/167; A61K 31/4704; A61K 31/44; A61K 31/538; A61K 31/138; A61K 31/55; A61K 31/439; A61K 31/5386; A61K 31/40; A61K 31/573; A61P 11/00

(57) ABSTRACT

This invention disclosed new use of R-terbutaline and other R-enantiomer β2-agonists as immune-modulators for treatment of bronchia-lung inflammatory symptoms or inflammatory fibrosis remolding. This invention also disclosed new use of R-terbutaline and R-β2-agonists for reduced adverse effects related to racemic or S-enantiomer β2-agonists including airway hyper responsiveness and airway fibrosis.

### CROSS-REFERENCE TO RELATED APPLICATIONS

This application claims the benefit of U.S. Provisional Application No. 62/844,398, filed May. 7,2019.

This application is also a continuation-in-part of International Application No. PCT/US19/50120, filed Sep. 7, 2019, which is a continuation of U.S. Application No. 62/728,800, filed Sep. 9, 2018.

### FIELD OF INVENTION

This invention disclosed new uses of R-enantiomer terbutaline and other R-enantiomer β2-agonists as immune-modulator for treatment of bronchia-lung inflammatory symptoms and/or inflammatory remodeling with reduce adverse effects. The adverse effects related to frequent use of racemic β2-agonists.

### BACKGROUND OF INVENTION

Terbutaline, a β2-agonist has been used for more than 30 years in treatment mainly asthma and also in some countries as tocolytic therapy for preterm labor. Terbutaline molecule consist of both R- and S enantiomers. Currently, marketed terbutaline as API in tablet, in aerosol or in injectable formula are racemic which contain 50% of S and R enantiomer terbutaline. It has been reported that R or S β2 agonist such as salbutamol could have very different in biological effects. R enantiomer is the utemor which are theoretically effective, while the S enantiomer is the distemor which does not activating β2 receptors and also toxic. Therefore, chiral switch could be beneficial for the patient in need. However, there few study about the adverse effects of S-terbutaline in prior art. Rather, It general believes that S-terbutaline is not toxic although it is therapeutically inactive, and therefore, using R-terbutaline instead of racemic terbutaline is not beneficial for patients in need. For this reason, it is believed that to develop R enantiomer terbutaline as inhaler or other formulas was not necessary since the high cost of R &D does not justify the clinic benefits to the patient. Nowadays, most β2 agonists on the market are still in its racemic form except few such as levosalbutamol and arformoterol. Major clinic trials demonstrated that there was no significant benefit for using inhaler β2 agonist over racemic. (Mansfield et al., Single-enantiomer drugs: elegant science, disappointing effects, Clinical Pharmacokinetics 43(5):287-90⋅February 2004)

On the other hand, there is increased concerns about the safety of long term use of β2 agonists because of airway hyper-responsiveness. Pulmonary deterioration and decreased long term survival rate are related to long term use of β2-agonists. In some cases, long-acting beta agonists (LABAs) have been linked to life-threatening asthma attacks and acute respiratory distress syndrome, ARDS) (Bassford et al. The rise and fall of β-agonists in the treatment of ARDS, Critical Care 2012, 16:208). Recently, it was suggested that instead of β2 agonists, β2-blockers may be benefit asthma patients. (Albouaini. K at el., Beta-blockers use in patients with chronic obstructive pulmonary disease and concomitant cardiovascular conditions, Int J Chron Obstruct Pulmon Dis. 2007 December; 2(4): 535-540)

This controversy causes problems in clinic management of asthma-COPD or ARDS. In this invention, we disclosed that there are major differences of R vs S terbutaline and other beta 2 agonists such R-salbutamol in term of efficacy and toxicity in treatment of asthma-COPD, respiratory failure and other life threatening conditions. The adverse effects of racemic β2 agonists may be related only to the S-enantiomers of β2 agonists. Therefore, using of R-terbutaline instead of racemic terbutaline could bring significant benefits to a patient in need. The same holds true for using other R-enantiomer β2 agonists.(salbutamol, bambuterol, vilanterol, salmeterol, clenterol, formoterol, trantinterol, Indacaterol etc.)

The other important aspect for using β2 agonists is that there is a long-term common understanding in medical practice that β2 agonists only used for relieving bronchospasm by relaxation of bronchiole smooth muscle. Corticosteroids are commonly used as anti-inflammatory agents in treatment of inflammatory lung symptoms, sometimes in combination with β2 agonists, while β2 agonists along have not been used for treatment inflammatory lung diseases or symptoms either related or not related to asthma/COPD. The effects of salbutamol on inhibiting some cytokines in animal or cellular studies were reported in prior art, but besides smooth muscle relaxation, β2 agonists were not considered by person in art to have therapeutic effects against immuno-inflammation or bronchial-fibrosis remodeling.

On these basis, evaluation of b2-AR agonists as potential anti-inflammatory drugs is strongly warranted. However, the relative contribution of b2-AR agonists, used as bronchodilation agents in asthma, to any eventual immune-modulating activity of these drugs to their overall therapeutic effect remains to be established.

In addition, the possible contradictory effects between R and S enantiomer β2 agonist were not well documented in clinic. When racemic β2 agonists were used, the possible therapy effects of R-enantiomer may be reduced or diminished by the contradictory acts of its S-enantiomer.

Repeated allergic reactions in response to pathogen associated molecular pattern (PAMP) often lead to “remodeling” processes which may be variable in severity and in proportion of changes from case to case. Principal among these changes are airway fibrosis, an elevation in smooth muscle mass, mucous metaplasia, and glandular hypertrophy, in addition to less well defined alterations of the bronchial vasculature and nerves to create an abnormal airway wall.

The invention disclosed new uses of R-enantiomer β2 agonist as anti-immuno-inflammatory medicine to prevent or treatment lung inflammation and exacerbated chronic bronchiolitis, brachialis-lung fibrosis remodeling, emphysema, and excessive mucus production induced by immune-inflammatory responses to pathogen-associated molecule pattern (PAMP) in the cases of allergic reactions, cytokine storm in sepsis or acetylcholine storm. Furthermore, this invention disclosed new use of R-enantiomer β2 agonist to reduced adverse effects which related to long term use of racemic β2 agonist (which consists of 50& S-enantiomer) including chronic bronchiolitis, chronic obstructive pulmonary disease, lung fibrosis and pulmonary hypertension.

### DETAILS OF INVENTION

In this invention, OVA-sensitized/OVA-challenged mice were used to study the effects of R, S or racemic β2 agonist in order to mimic clinic allergic reactions. The animals manifested severe allergic inflammatory symptoms, including increased airway resistance, immune-inflammatory response, cytokine release, ROS production and patho-histological changes in lung tissue. After the allergic asthmatic model was established by OVA immunization and challenges, the animals were pre-treated with aerosol of drugs above repeatedly for 7 days by inhalation. In the same animal, asthmatic attack was evoked by inhalation of methacholine and lung function were studied. BALF and tissue samples were collected for analysis. These protocol is similar to clinic treatment, in which the asthma patients were treated with drugs repeatedly, The baseline conditions were very much different in comparing normal animal used in prior art. It is commonly known that the responses to β2 agonists in animal with pre-existed asthmatic conditions are fundamentally different than the responses seen in normal animal or in cultured cell-lines used in prior art. The results based on asthma animals disclosed by this invention cannot be anticipated by a person in art according to results from normal animals or in cultured cells in prior art.

This invention disclosed that S-enantiomer enhanced airway resistance and responsiveness in OVA-sensitized/OVA-challenged mice asthmas. In ovalbumin (OVA) induced asthma mice, Treatment of S-terbutaline significantly enhanced the baseline airway resistance and reduced lung compliances. When the asthmatic mice were challenged with methacholine, a muscarinic receptor agonist, the asthma responses were much severe in S-enantiomer pretreated mice than either R-enantiomer or Saline (control) pretreated mice. The increase of airway resistances as measured by plethysmography were significant higher in S-enantiomer pretreated mice in comparison of either saline or R-enantiomer terbutaline pretreated mice. A severe constriction of bronchiole can be observed under microscopy in lung tissue section in S-terbutaline treated asthmatic mice. While, R-enantiomer terbutaline is significantly more effective against in anti-asthma and inflammatory pathology than RS terbutaline. The same holds true for R or S-enantiomer of Salbutamol, bambuterol, vilanterol, clenbuterol, salmeterol, formoterol, trantinterol and Indacaterol.

This demonstrated clearly that S-enantiomer is not only therapeutically inactive, but responsible for drug induced airway hyper responsiveness. This invention disclosed that S-enantiomer is link to life-threatening asthma attacks in patient receive racemic β2 agonist treatments. Therefore, use of R-enantiomer instead of RS-racemic can reduce unwanted adverse effects.

This invention disclosed that S-enantiomer β2 agonists increased eosinophil in blood and in lung infiltration as measured in bronchi-alveolar lavage fluid. In asthmatic mice eosinophil is important marker for asthma response which can mediates severe inflammatory responses. However, in prior art, it was reported that in OVA-sensitized/OVA-challenged mice, R-salbutamol did not significantly reduce eosinophil. In other prior art, it reported that both (R)- and (S)-enantiomers of salbutamol reduced airway eosinophil trafficking and mucus hypersecretion in a mouse model of asthma. (by Henderson et al. Journal of Allergy and Clinical Immunology 116(2):332-40⋅ September 2005). In this invention, S-terbutaline and other S-enantiomer beta2 agonist were inhaled and exposed every day for period of 7 days in mice after OVA-sensitized/OVA-challenged asthma, in order to mimic clinic treatment. While, in the prior art, S-salbutamol was given parentally by implanted osmic pump. The controversy may due to different rout of drug administration.

This invention disclosed that counts of eosinophils in either blood or BALF were significantly increased after repeatedly inhale of S-terbutaline in OVA-induce asthma mice. Inhalation of racemic terbutaline has less but similar effects as S-terbutaline, while repeated inhalation of R-terbutaline has opposite effects comparing S or racemic terbutaline, the counts of eosinophil in both blood and BAL were significantly reduced in asthma mice. The same were also found in racemic, R or S-enantiomer of following: Salbutamol, bambuterol, vilanterol, clenterol, salmeterol, formoterol, trantinterol and Indacaterol and other β2 agonists.

This disclosure by this invention demonstrate that R-terbutaline and other β2 enantiomer agonists can be used for reducing eosinophil mediated asthma responses, while S-terbutaline and other β2 5-enantiomer agonists can worsen the eosinophil mediated asthma responses when inhaled by patient in need. For the same reason, inhalation of racemic β2 agonist which contain 50% of S-enantiomer could be harmful for asthmatic treatment in patient. This has never been disclosed by prior art, in fact, the prior art disclosed the opposite, Therefore, the disclosure of this invention should be considered as novel and inventive.

This invention disclosed that S-enantiomer β2 agonist enhances cytokines and ILC mediated inflammation in bronchio-alveolar lavage fluid (BALF) . There was an activation of innate lymphocytes (ILC) and type 2 inflammation and increased release of IL4 and IL13 in BALF in OVA-induce asthma mice. This invention disclosed that after repeated inhalation of S-terbutaline and other S-enantiomer β2 agonists for a long period of time, there was further increase of cytokines including: IL4 and IL13 in BALF and IgE in blood in AVO-sensitized/AVO-challenged asthma mice. Increase of these cytokines indicates an activation of innate lymphocytes (ILC) in the lung. On the other hand, repeated inhalation of R-terbutaline and other R-enantiomer β2 agonists significantly inhibited the release of IL5 in BALF in OVA-sensitized/OVA-challenged mice OVA-sensitized/OVA-challenged asthma mice. Similar results were also found in racemic, R or S-enantiomer of salbutamol, bambuterol, vilanterol, clenterol, salmeterol, formoterol, trantinterol and Indacaterol and other β2 agonists. It is reported in prior art that racemic β2 agonists salbutamol and clenterol may inhibit the activation of ILC in the lung. This invention disclosed that S-enantiomer β2 agonist is, in fact, activating type 2 inflammatory and ILC in lungs. The opposite effects on type 2 inflammation between S and R enantiomer salbutamol and clenterol disclosed in this invention were not report in prior art. This invention disclosed that use of R-enantiomer instead of racemic in this case would bring unexpected and significant benefits in managing lung inflammation to a patient in need. This disclosure in this invention should be considered novel and inventive.

Persistent bronchia-pulmonary inflammatory would lead to inflammatory remodeling. This invention disclosed that repeated inhalation of S-enantiomer β2 agonist can further enhance the hypertrophy and proliferation of bronchiole smooth muscle and lung fibroblasts cells, and deposition of collagen in extracellular matrix in OVA-sensitized/ova-challenged mice. In OVA-sensitized/ova-challenged asthma mice developed severe inflammation, mucosal edema and epithelial lesions, interstitial infiltrates of neutrophils, lymphocytes and eosinophils. The hypertrophy and proliferation of smooth muscle and fibroblasts cells resulted in increased thickness of the bronchiole smooth muscle layer and collagen deposition. When S-terbutaline was inhaled repeatedly for days, the above histopathological changes are further worsened, along with severe constriction of the lumen of bronchioles filled with over production of mucus. Therefore, the inflammatory remodeling was enhanced by S-terbutaline, but diminished by R-terbutaline.

There was also a further increase of thickness of alveolar septum in lung tissue. These would result in deterioration in lung function and decrease in oxygen exchange via gas-blood barrier in OVA-sensitized/ova-challenged asthma mice.

Repeated inhalation of S-terbutaline increased goblet cell hyperplasia and excessive mucus production in OVA-sensitized/OVA-challenged mice. PAS and Masson staining displayed that the hyperplasia of airway goblet cells and collagen deposition and increased sub-epithelial matrix glycoproteins. These would lead to exacerbation of bronchia-lung inflammation. lung fibrosis, pneumonia and emphysema, COPD or atelectasis.

On the other hand, inhalation of R-terbutaline inhibited showed the opposite effects as its S-enantiomer. R-terbutaline significantly inhibited the proliferation of bronchiole smooth muscle cells, fibroblasts and collagen deposition, goblet cell hyperplasia and mucus production in the asthma mice. Inhalation of racemic terbutaline was much less or not effective in comparing R-terbutaline.

Similar effects or results also found in racemic, R or S-enantiomer beta2 agonists including: salbutamol, bambuterol, vilanterol, clenterol, salmeterol, formoterol, trantinterol and indacaterol and other β2 agonists.

It was reported that S-salbutamol could increase the proliferation of bronchiole smooth muscle and fibroblast in normal cell culture preparation. This was only based on measuring total proteins in the culture dish rather than counting the cells directly. (by Basil O Ibe, et. al, Int Arch Allergy Immunol 2008; 146:321-333). It didn't teach which type of cells were proliferated, and also didn't teach whether the protein measured was due to increased secretion activity of individual cell or due to increased cell numbers. On the other hand, this invention disclosed the effects of S-terbutaline or S-salbutamol on a disease, in vivo, animal model under inflammatory, rather than normal, conditions by OVA sensitize/challenge, and the drugs were tested by inhalation into lungs. Furthermore, this invention disclosed modifications of the OVA sensitize/challenge evoked inflammation by repeated pretreatment of S-terbutaline or S-salbutamol or other beta2 agonists. Therefore, the prior art shouldn't render the disclosure of this invention obvious.

Vascular changes are important part of lung inflammatory remodeling. This invention disclosed that inhalation of S-enantiomer β2 agonists further increased of the intermedia layer of blood vessels in OVA sensitive/ova challenged mice. In the lungs of OVA sensitive/ova challenged mice, there were increased size and number of vascular smooth muscle cells and fibroblasts, excessive collagen deposition and inflammatory infiltration in the vascular interstitial matrix. The endothelia cells are proliferated and distorted in shape. The vascular wall thickness was increased. After repeated inhalation of S-terbutaline, there were further enhanced proliferation of endothelia, smooth muscle and fibroblast cells, and more deposition of collagen in the intermedia layer of vessels, which resulted in further increase of wall thickness of vessels. In addition, there was proliferation of fibroblasts and collagen deposition in interstitial layer of alveolar and increased thickness of septum of alveolar. These changes may lead to reduction of oxygen exchange, increase in vascular resistance and result pulmonary hypertension. Therefore, this invention disclosed that inflammatory remodeling processes as shown in OVA sensitive/ova challenge were further augmented after repeated inhalation of S-terbutaline, while these processes were significantly diminished after inhalation of R-terbutaline. The disclosure of this invention cannot be anticipated by an person in art. It should be considered as novel and inventive, This invention also disclosed similar effects or results for use of other racemic, R or S-enantiomer beta2 agonists including: salbutamol, bambuterol vilanterol, clenterol, salmeterol, formoterol, trantinterol and Indacaterol and other β2 agonists.

Respiratory failure such as acute respiratory distress symptom can be induced by sepsis. It was characterized with a rapid and excessive release of cytokines, chemokines and immune-dysfunction in response to virus, bacteria or PAMP such as PLS. It is well accepted that macrophage activation is a critical and important step in sepsis. This invention disclosed that S-albuterol (salbutamol) facilitated macrophage polarization, increased the release of cytokines and the production of reactive oxygen species (ROS) during inflammatory challenges by PLS, while R-salbutamol significantly inhibited the polarization of macrophages. (R)-salbutamol inhibited the release and expression of cytokines in LPS induced M1 macrophages, including TNF-α, IL-1β, IL-4,5,6,13 and MCP-1. These inhibitory effects can be blocked by ICI-118551, a specific β2 agonist blocker. S-salbutamol had the opposite effects. Macrophages play key roles in the initiation of severe allergic response, and cytokine storms, respiratory distress or failure induced by sepsis. M1 macrophage polarization promotes inflammatory disease. In this study, we discovered that (R)-salbutamol, a β2 receptor agonist, inhibits and reprograms the cellular metabolism of macrophages. (R)-Salbutamol significantly inhibited LPS-induced M1 macrophage polarization, as determined by flow cytometric analysis. (R)-Salbutamol downregulated both the protein and mRNA expression of typical M1 macrophage cytokines, such as tumor necrosis factor α (TNF-α), interleukin-1β (IL-1β) and monocyte chemotactic protein-1 (MCP-1), suggesting that (R)-salbutamol could transcriptionally suppress cytokines typically expressed in M1 macrophages.

Furthermore, (R)-salbutamol increased the ratio of reduced glutathione (GSH)/oxidized glutathione (GSSG), inhibited inducible nitric oxide synthase (iNOS) and significantly decreased the production of nitric oxide (NO) and reactive oxygen species (ROS); in contrast, (S)-salbutamol increased the production of NO and ROS. Bioenergetics profiles showed that (R)-salbutamol significantly reduced aerobic glycolysis and attenuated mitochondrial respiration. Moreover, untargeted metabolomics analysis demonstrated that (R)-salbutamol modulated metabolic pathways; phenylalanine metabolism, the pentose phosphate pathway and glycerophospholipid metabolism were the most markedly impacted pathways by (R)-salbutamol. The effects of (R)-salbutamol on M1 polarization were blocked by a specific β2 receptor antagonist, ICI-118551. This invention disclosed that (R)-salbutamol inhibits the M1 phenotype of LPS-induced macrophages by downregulating aerobic glycolysis and glycerophospholipid metabolism. This disclosure by this invention is novel and inventive.

Macrophage activation and proliferation was considered to be the key step of initiation and development of sepsis. In one embodiment, LPS (PAMPs from bacteria) induced a proliferation and polarization of macrophages from the peritoneal fluid. While the number of these macrophages significantly reduced toward normal level when treated with (R)-salbutamol. There was also significant reduction of polarized M1 macrophages or M2 macrophages in comparison of sepsis condition. These inhibitory effects can be blocked by using specific β2-recepter blocker. On the other hand, treatment of sepsis with (S)-salmeterol, both M1 and M2 macrophages were further increased in comparison of sepsis condition.

LPS interacts with pathogen recognition receptors such as TLRs would also activate NF-κB and MARK pathways, which would further lead to an over-production of cytokines. This invention disclosed that (R)-terbutaline significantly inhibited both of NF-κB and MARK pathways activated by LPS in sepsis, by reducing their expression or phosphorylation. However, in the contrary, (S)-terbutaline further enhanced both NF-κB and MARK pathways in sepsis. The “cytokine storm” in sepsis involves in excessive release of INFs, IL-1B, IL-2,4,6,10,12, TNFa and MCP etc. This invention disclosed that (R)-terbutaline as well as (R)-salbutamol can effectively prevent or ameliorate the “cytokine storm” and significantly reduced the production or release of these cytokines.

In sepsis, there are also over productions of chemokines such as ROS. This invention disclosed that (R)-terbutaline and (R)-salbutamol could significantly prevent and inhibit the over production of inducible NO (iNO) and another ROS induced by LPS sepsis. This invention disclosed the similar effects of other RS-racemic, S-enantiomer or R-enantiomer beta2 agonists including: salbutamol, bambuterol, vilanterol, clenterol, salmeterol, formoterol, trantinterol and Indacaterol and other β2 agonists.

Sepsis has relatively high rate of mortality. It is mainly due to an imbalanced host immune responses to PAMP and excessive production of cytokines and chemokines, which lead to a lung inflammation with respiratory distress or multiple organ failure. Macrophage activation is one of the most important step in development of sepsis. This invention disclosed that the rate of mortality of sepsis was greatly reduced or even diminished by (R)-terbutaline and (R)-salbutamol. It was probably through inhibiting the activation and proliferation of macrophages. The therapeutically effects of (R)-terbutaline and (R)-salbutamol against macrophage activation, inhibiting various cytokines and chemokines, and alleviating sepsis cannot be anticipated from prior art.

This invention also disclosed similar effects of other short or long acting (R)-enantiomer beta2 agonists including: salbutamol, bambuterol, vilanterol, clenterol, salmeterol, formoterol, trantinterol and Indacaterol.

Combination therapy is commonly used in clinic management of bronchia-lung inflammatory symptoms. This invention disclosed beneficial effects of combining muscarinic receptor agonist with R-enantiomer β2 agonist instead of its racemic or S-enantiomer. In ova-sensitized/ova-challenged mice, inhalation of S-salbutamol results in more severe inflammation and tissue remodeling reactions as indicated above. When ipratropium, a muscarinic receptor antagonist was combined with S-salbutamol, there was no observable improvements in asthma mice. However, When R-salbutamol was used instead of S-enantiomer, the above asthmatic conditions was significantly improved. In particularly, the proliferation of goblet cells and mucus overproduction were diminished significantly, Epithelia cells at the inner layer of bronchiole were restored. In addition, the airway resistance evoked by methacholine is much greater in S-salbutamol treated asthma mice comparing saline (control) treated asthma mice. The same were also found in racemic, R or S-enantiomer of terbutaline, bambuterol, vilanterol, clenterol, salmeterol, formoterol, trantinterol and Indacaterol and other β2 agonists. Therefore, this invention disclosed that in the combination therapy of β2 agonist with ipratropium or other muscarinic receptor antagonist, using R-enantiomer instead of racemic results in significant better effects and much more reduced adverse effects.

This invention disclosed that similar beneficial effects were also found when corticosteroids such as budesonide or fluticasone propionate was used instead of muscarinic receptor antagonists.

This beneficial effect of R-enantiomer-β2 agonist in combination therapy has not been disclosed by prior art. It should be considered as novel and inventive.

Furthermore, this invention disclosed that in a triple combination therapy, in which a muscarinic antagonist (ipratropium), a corticosteroid (budesonide or fluticasone) propionate and a R-enantiomer β2 agonist (R-terbutaline or R-salbutamol or R-salmeterol) were used in combination. In the triple combination, R-enantiomer β2 agonist was superior over its racemic against asthmatic attack, immune-inflammation and tissue remodeling.

The muscarinic receptor antagonist according to this invention including tiotropium, glycopyrronium, aclidinium and umeclidiniumt.

The corticosteroids according to this invention include ciclesonide, beclomethasone, mometasone, dexamethasone, prednisolone, flunisolide, triamcinolone acetonide and other pharmaceutically used corticosteroids or their physiological acceptable salts and/or solvate thereof according to this invention.

It is common understanding in medical practice that β2 agonists are used only for anti-bronchospasm by relaxation of bronchiole smooth muscle. It has not been used as immuno-modulator for treatment of inflammatory lung diseases or symptoms.

The inhibition of some cytokines in BALF or cultured cells by R-salbutamol have been reported, but in prior art, β2 agonist have never been recommended for used as anti-bronchia-lung inflammatory medicine besides as bronchodilator.

This invention disclosed new uses of R-enantiomer β2 agonist as anti-immuno-inflammatory medicine to prevent or treatment bronchia-lung inflammation and inflammatory remodeling. This invention disclosed that R-enantiomer β2 agonists can also be used to treat lung immuno-inflammatory conditions which related to other diseases such as IBD (inflammatory Bowel Diseases), HIV infections, tuberculosis, idiopathic pneumonia, interstitial lunge diseases (ILD), idiopathic pulmonary fibrosis (IPF), extrinsic allergic alveolitis (EAA), lung cancer or septic shock and systemic infection.

Furthermore, this invention disclosed new use of R-enantiomer β2 agonist for reduced adverse effects which related to long term use of racemic β2 agonist including persistent and exacerbated chronic bronchiolitis, deteriorated chronic obstructive pulmonary disease, lung fibrosis and pulmonary hypertension.

This invention disclosed similar beneficial effects of other R-enantiomers β2 agonists includes: R or R′R′ enantiomers of Short-acting β2 agonists: bitolterol, fenoterol, isoprenaline, orciprenaline ormetaproterenol, pirbuterol, procaterol, ritodrine; and Long-acting β2 agonists,: bambuterol, arformoterol, Formoterol, Perforomist; and Ultra-long-acting β2 agonists: abediterol, carmoterol, indacaterol, Olodaterol, vilanterol, isoxsuprine, mabuterol and zilpaterol.

The pharmaceutical preparation of R-terbutaline and other R-enantiomer β2 agonists as listed above according to this invention include solid dose forms for oral use; gel, suppository for rectum or vagina, eye drop, liquid or lyophilized powder for injection; ointment or patches for topic use and aerosol or dry powders for inhalation into lung and nasal.

The pharmaceutical acceptable salts of R-terbutaline and other R-enantiomer β2 agonists as listed above according to the invention include those formed with conventional pharmaceutical acceptable inorganic or organic acids for example: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

### BRIEF DESCRIPTION OF DRAWINGS

FIG. 1 The response to aerosolized methacholine, a broncho-constrictor, in OVA-sensitize/challenged mice after seven days' pretreatment by inhalation of R-, S-, and RS terbutaline. This figure disclosed the adverse effects of hyper-responsiveness due to repeated use of S-enantiomer β2 agonists.

FIG. 2 The lung histopathologic changes in OVA-sensitize/challenged mice as descripted in FIG. 1.

FIG. 3 Cytokines released after pretreatment with R-, S- and racemic terbutaline for 7 consecutive days in OVA-sensitize/challenged mice. R-terbutaline significantly inhibited the release, but S-terbutaline did the opposite.

FIG. 4 The phosphorylation and activation of MAPK and NF-κB pathway affected by pretreatment of R. S- and racemic terbutaline. R-terbutaline decreased the both of ratios of phosphorylated to total p38 MAPK; and phosphorylated to total p65 NF-κB, while S-terbutaline enhanced both of the ratios. R-terbutaline was anti-inflammatory, but S-terbutaline was pro-inflammatory.

FIG. 5. Periodic acid Schiff (Paration S) stain of lung tissue from the mice of FIG. 2. This figure disclosed the processes of bronchio-lung inflammatory and inflammatory remodeling affected by pretreatment of R-,S, and RS-terbutaline.

FIGS. 6A and B. Masson stain and Periodic acid Schiff (Paration S) stain of lung tissues form normal and OVA-sensitize/challenged mice with pretreatment of R-salbutamol (R-Sa), S-salbutamol(S-sa), and combination with Ipratropium (IPR). The anti-inflammatory effects of R-salbutamol were enhanced by combination with muscarinic blocker.

FIG. 7 (R)-salbutamol inhibited the activation and polarization of M1 macrophages induced by LPS-challenges. Macrophages activation is a key step in sepsis induced respiratory failure.

FIG. 8 (R)-salbutamol inhibited the expression of cytokines in M1 macrophage induced by LPS-challenges.

FIG. 9 (R)-salbutamol inhibited the expression of iNOS and NO production in macrophages induced by LPS-challenges. iNOS and NO are important chemokines involved in inflammatory processes.

### EXAMPLES

### Example 1

OVA sensitized/challenged asthma model Mice were sensitized and challenged with ovalbumin (OVA) or normal saline. Briefly, mice were sensitized by intraperitoneal (i.p.) injection of 0.2 mL of 2% aluminum hydroxide (ALUM) gel containing 10 μg of OVA antigen on days 0 and 14. On days 21, 22 and 23, the mice were challenged with 1% OVA in saline (0.01 g/mL) for 20 min via. a nebulizer (PART Turbo boy). On day 26, a 20 min nebulized OVA challenge with 5% OVA in saline (0.05 g/mL) was conducted. The control group received 0.2 mL of saline with 2% ALUM administered intraperitoneally on days 0 and 14 before receiving 20 min nebulized normal saline without OVA on days 21, 22, 23 and 26.

### Example 2

### Methods:

### Measurement of Airway Responsiveness to Methacholine

In vivo airway responsiveness to methacholine (Mch) was assessed on day 28 in conscious, freely moving, spontaneously breathing mice by a whole-body plethysmography (Buxco Electronics Inc.). Mice were challenged with aerosolized saline or increasing concentrations of Mch (2, 10, 20 mg/mL) administered by an ultrasonic nebulizer for 2 min. Before Mch challenge, a 20 μL drug aerosol or saline was given to the mice because of the short-acting bronchodilating effect of terbutaline. The degree of bronchoconstriction was expressed as enhanced pause (Penh), a calculated dimensionless value that correlates with measurement of airway resistance, impedance, and intrapleural pressure. Penh values were recorded and averaged for 4 min after each nebulization challenge.

### Sample Collection and Whole Blood Analysis

After Methacholine challenge, mice were anesthetized, blood was collected for hematology analysis. Part of the collected blood was transferred to a heparinized Eppendorf tube, gently mixed and centrifuged under 845×g at 4° C. for 10 min to isolate plasma specimen for further analysis. Broncho alveolar lavage (BAL) was performed on the right lung using 0.3 mL of phosphate buffered saline (PBS) for four times. The saline was instilled into the lungs and allowed to equilibrate for 30 s before recovery, after centrifugation supernatants were stored at −80° C. for further cytokines analysis while cell pellet was suspended in 0.5 ml PBS and the eosinophils counts were performed using Wright's stain. Then all the organs (liver, right lung lobe, spleen, kidneys, heart, brain) were excised surgically and washed totally with ice-cold PBS to clear the blood and then blotted dry with filter paper. These tissue specimens were then accurately weighted and stored at −80° C. for detecting the drug distribution by LC-MS/MS. White blood cell (WBC) counts and differential WBC counts were analyzed using a ProCyte Dx Hematology Analyzer (IDEXX Laboratories) in 4 h after blood collection.

### Measurement of OVA-sIgE

OVA-sIgE in plasma was measured using an enzyme-linked immunosorbent assay (ELISA) kit following the manufacturer's specification.

### Analysis of Cytokine Levels in BAL Fluid

BAL fluid samples were analyzed for the concentrations of mouse IL-4 and IL-5 using commercial ELISA kits according to the manufacturers instructions (R&D Systems, Minneapolis, Minn.). IL-13 was assayed in BAL fluid with a mouse IL-13 immunoassay kit (Abeam, Cambridge, UK) following the manufacturer's protocol.

### Lung Histopathology

Left lung tissue was obtained for histopathology and fixed in 10% formalin for 24 h, followed by paraffin-embedded and then were sectioned at 4 μm for routine staining with hematoxylin and eosin (H&E, Solarbio, Beijing, China), or with periodic acid-Schiff (PAS, Solarbio, Beijing, China), or submitted to Masson's trichrome staining (Baso, Zhuhai, China). Images of the stained lung sections were obtained by an Axisplus image-capturing system (Zeiss, Germany) and then a minimum of 3 bronchi (luminal diameter, 150-500 μm) were analyzed per mice for various parameters using an image analyzing computer system (IPP) by pathologists blinded to grouping. Histological score, airway smooth muscle thickness, PAS score (goblet cells hyperplasia), and collagen deposition were evaluated by a semi-quantitative scoring method.

The level of mRNA of NF-κB in the lung were analyzed by quantitative real-time PCR analysis. The total RNA was extracted from lung tissue using RNaEX™ Total RNA Isolation Kit (Generay Biotech). The quality and quantity of RNA samples were assessed by NanoDrop ND-2000c spectrophotometer (Thermo Fisher Scientific, Inc., Wilmington, Del.). Total RNA (1 μg) was reverse transcribed into cDNA using cDNA Reverse Transcription Kit (Vazyme Biotech, Nanjing, China) according to the manufacturer's instruction. Real-time PCR reactions were performed using the 7500 real-time-PCR system (Applied Biosystems, Foster City, Calif., USA) with AceQ qPCR SYBR Green Master Mix (Vazyme Biotech, Nanjing, China). The primers, set for mouse, were synthesized by Sangon Biotech (Shanghai, China).

### Western Blot Analysis

Total protein from lung tissue was extracted according to the guidelines of manufacturer (Beyotime, China) and the concentrations were determined by BCA assay (Pierce). Primary antibodies against p38 MAPK (CST #8690), p-p38 MAPK (Thr180/Tyr182, CST #4511), NF-κB p65 (GeneTex #GTX102090), p-NF-κB p65 (S536, CST #3033) and GAPDH (Affinity #AF7021) as a standard were purchased and used.

### Results: Effects of R-, S- and rac (RS)-Terbutaline on OVA-Induced Airway Hyper Responsiveness

OVA-induced mouse model was established and airway reactivity was assessed in response to aerosolized Mch by means of noninvasive in vivo plethysmography. As shown in figure below, compared with the control group, the baseline value in OVA group was significantly augmented when exposure to saline (0.80±0.12 for OVA group vs 0.55±0.08 for Control group). R-terbutaline and double molar amount of rac-terbutaline treatment could significantly reduce the baseline Penh values. However, the baseline airway reactivity after S-terbutaline treatment was significantly increased (0.70±0.13) compared with the control group. Moreover, S-terbutaline treatment group showed an further exacerbation of airway hyperactivity in response to the increased doses of Mch. Treatment of R-terbutaline attenuated significantly airway bronchoconstriction to Mch and showed effective bronchospasm protection. While RS-terbutaline (rac-ter) (double dose of R-ter) administration exhibited much less effect against Mch in comparing with R-ter/OVA group.

### Whole Blood Analysis and Eosinophils Counts in BAL Fluid

Total WBCs and differential WBCs (Neutrophilis, lymphocytes, monocytes, eosinophils and basophils) in blood and eosinophils counts in BAL fluid were detected. Total WBCs in blood were similar without significant difference. The percentage of differential WBCs in blood were found to be no significant difference within all the groups besides the percentage of eosinophils counts. The per cent of eosinophil counts in blood and BALF was significantly higher in OVA, S-terbutaline and RS(rac)-terbutaline treated groups comparing with control group. The eosinophil counts were the highest in S-terbutaline group. The Treatment with R-terbutaline significantly inhibited the influx of eosinophils into BALF.

### Histopathological Evaluation of Lung Tissue

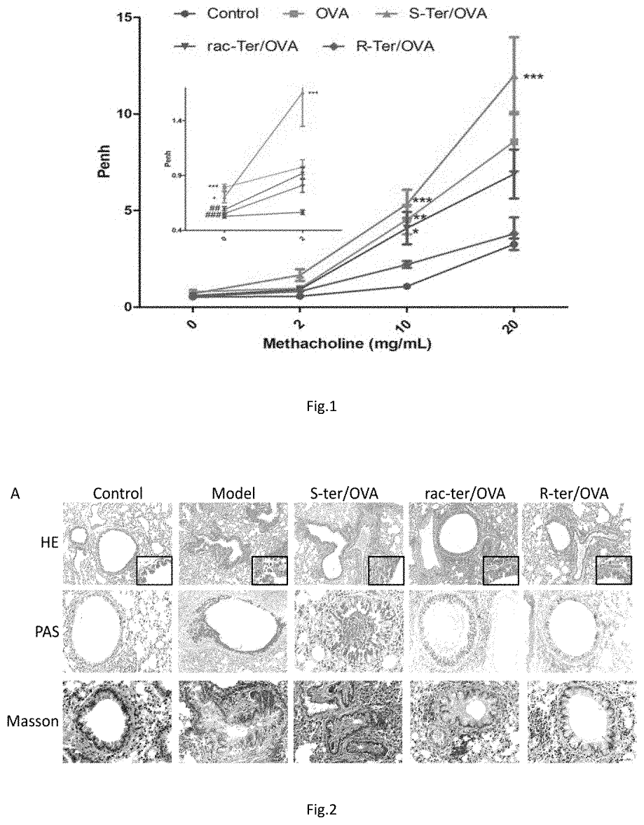

To further investigate the allergic inflammatory changes in the lung of mice, H&E staining was then conducted. As shown in FIG. 2A, no inflammation, mucosal edema and epithelial lesions were observed in the control group, whereas OVA-induced asthma mice developed severe inflammation, mucosal edema and epithelial lesions, which included interstitial infiltrates and a large number of lymphocyte and eosinophil infiltration (FIG. 2B model). With R-terbutaline and double dose of rac-terbutaline treatment, the degree of inflammatory cell infiltration was ameliorated respectively, which was confirmed by the histological score. Meanwhile, OVA-treated mice exhibited smooth muscle hypertrophy in both bronchioles and vascular vessels, increased thickness of bronchial and vascular smooth muscle layer, which were suppressed by R-ter and further enhanced by S-ter (FIG. 2C). Similarly, PAS and Masson staining displayed that there were deposition of subepithelial matrix glycoproteins, hyperplasia of airway goblet cells and collagen deposition in OVA group and these conditions were further exacerbated after S-ter treatment. On the other hand, the inflammatory condition, collagen deposition, hyperplasia of goblets and proliferation and hypertrophy of smooth muscle and fibroblasts seen in OVA group were reversed and recovered toward normal after treatment of R-ter.

### OVA-sIgE (Serum IgE) and Th2 Cytokines Measurements

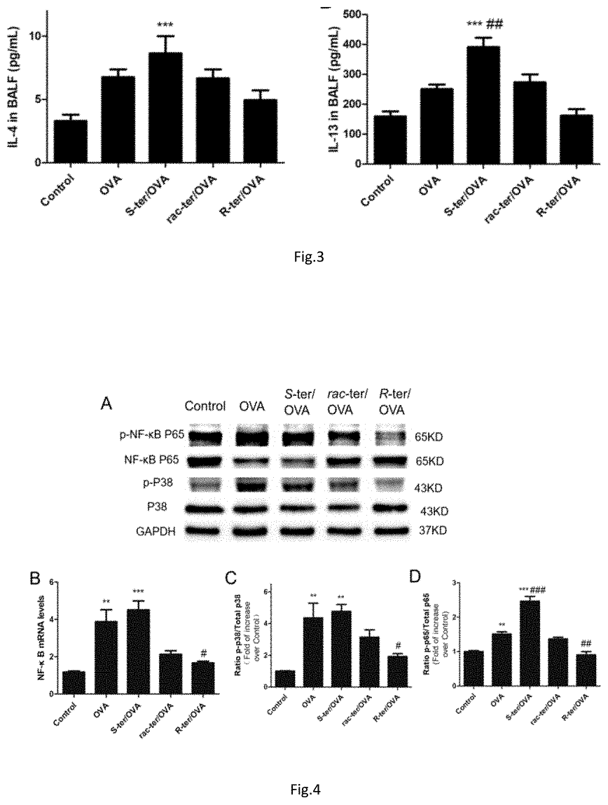

Plasma OVA-sIgE and representative Th2 cytokines (IL-4, IL-5, IL-13) in BAL fluid. were assessed. Mice treated with OVA displayed a significant increase in OVA-sIgE level, while markedly inhibited by administration of R-ter. Likewise, Th2 cytokines (IL-4, 5 and 13) were found to be augmented in OVA-treated group, R-ter-treatment significantly reduced IL-5 in BALF. However, S-ter treatment further increased IL-4 and IL-13 release comparing to OVA group (FIG. 3). This demonstrate that S-terbutaline can activate ILC and evoke Th2 type inflammation.

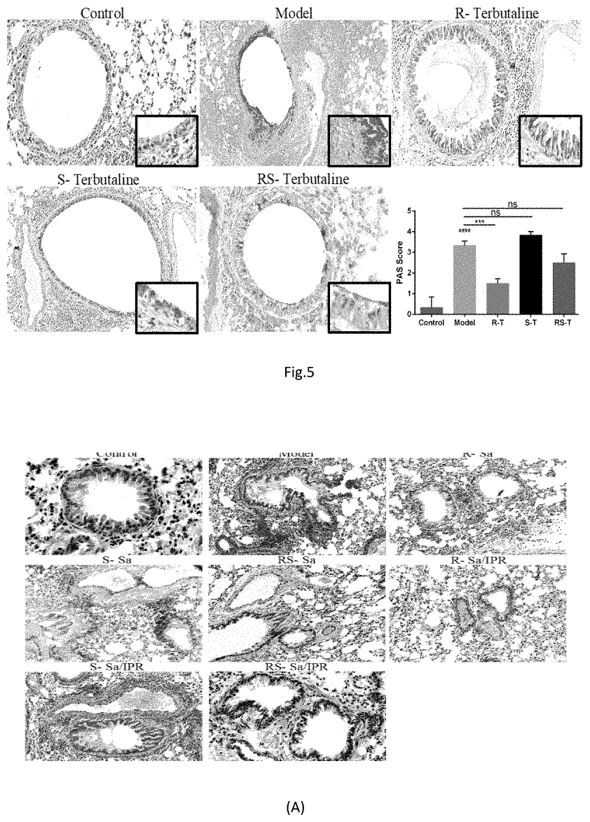

R-terbutaline inhibited p38 MAPK phosphorylation and NF-κB expression in lung, After challenges with OVA, the expression of inflammatory NF-κB was increased in mRNA transcript when compared with the control group (FIG. 4B). This was further enhanced in S-ter-treated group. On the other hand, R-ter treatment effectively inhibited the elevated NF-κB expression. Similar results have been seen in the phosphorylation of p65 subunit of NF-κB (FIG. 4D). MAPKs, especially p38 and ERK1/2, are involved in airway inflammation and regulation of the inflammatory mediators, such as the activation of NF-κB. Therefore, this invention disclosed he inhibition of inflammatory response by R-ter was mediated through MAPKs pathway in OVA-induced asthmatic mice, As shown in FIG. 4C, OVA-treated mice showed markedly upregulated p38 MAPK phosphorylation, S-ter treatment further increased p38 MAPK activation in comparing with OVA group, R-ter. treatment markedly suppressed the activation of p38 MAPK in comparing with OVA group. Collectively, these data revealed that R-ter exerted an anti-inflammatory effects on OVA-induced asthmatic mice through inhibiting the activation of p38 MAPK phosphorylation and NF-κB expression, which racemic RS-terbutaline (rac-ter) exerted much less effects. In the contrary, treatment of S-ter further enhanced p38 MAPK phosphorylation and NF-κB activation in comparing with saline treated group in ova sensitized asthma mice.

### Example 3

### Effects of R- and S-Terbutaline on PAS Stain of Lung Tissues

This invention disclosed that in OVA-sensitized/challenged asthma mice, there are significant inflammatory reactions and deteriorating in histopathological changes, including increased in connective tissue, mucin, fibroblasts, smooth muscles and collagen secretion and thickness of septum of alveolar and bronchiole walls. In addition, there were also clear constriction of bronchiole. These symptoms are further enhanced by S-terbutaline. Treatment of R-terbutaline can significantly ameliorate these changes.

There were significant increase of subepithelia matrix glycoproteins, collagen, fibroblast and inflammatory infiltrations (model), which were further worsened by S-terbutaline. These changes were significantly alleviated or improved by R-terbutaline. RS-terbutaline has significant less beneficial effects than its R-enantiomer. The changes of the area of collagen deposition as stained by PAS in each treatment groups were shown in figure,

### Example 4

Effects of Combination Therapy of Ipratropium with RS-R and S-Salbutamol

Methods used were similar as mentioned above. Nebulized aerosol of saline, Methacholine 2 mg/ml (low dose) or 10 mg/ml (high dose) were given via inhalation to OVA induced asthma mice.

### Results

1) Lung functions: Ipratropium (Ipr) is a muscarinic receptor blocker in combination with salbutamol(Sa), a beta2 agonist showed improved effects for anti-asthma and anti-inflammation. The increase in airway resistant in response to methacholine was significantly diminished by R-salbutamol, and the effect was further improved When combined with Ipratropium. The effects of R-salbutamol as combination therapy is much more effective that RS-salbutamol. However, S-salbutamol along could further enhance the airway response to methacholine. The adverse effects of S-salbutamol can be reduced by combine with ipratropium only in a small degree.
   Fig. increase in airway resistance after treatment's

| | |
|---|---|
| OVA + Saline | 445% |
| OVA + R-salbutamol | 180% |
| OVA + RS salbutamol | 214% |
| OVA + S-salbutamol | 486% |
| OVA + R-salbutamol + Ipra | 112% |
| OVA + RS-salbutamol + Ipra | 176% |
| OVA + S-salbutamol + Ipra | 313% |

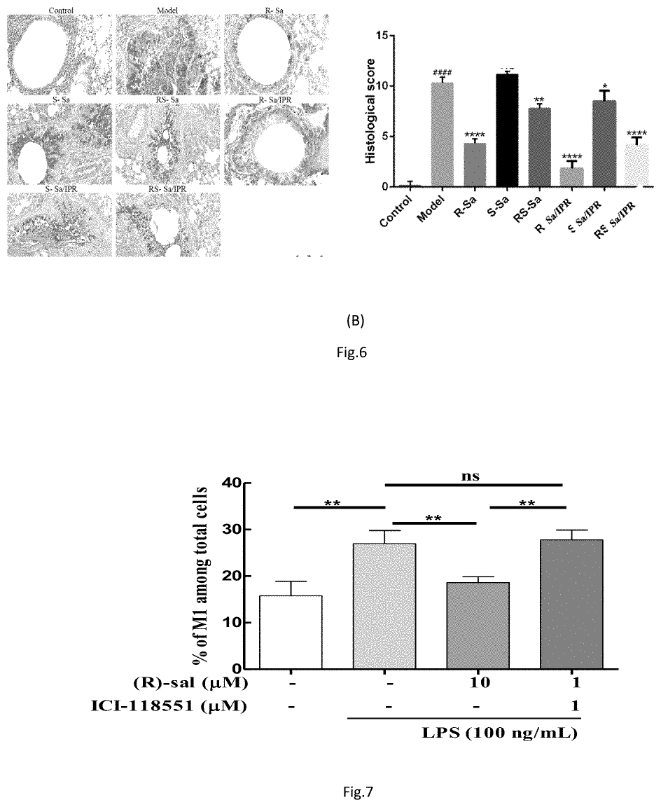

2) Lung histopathology:
   a. Masson staining
      There were increased collage depositions, fibroblasts and muscle cells in both bronchiole and vascular walls, with increased wall thickness S-salbutamol further worsens the inflammatory conditions and further increased the wall thickness of bronchiole and vascular vessels in comparing of ova (model) group. These adverse effects were reduced when combination with ipratropium. R-salbutamol showed significant therapeutic effects against the asthmatic responses. There beneficial effects were further enhanced by combine with ipratropium.
      FIG. 6B. In the Periodic acid Schiff (Paration S) stain staining, besides the similar changes noted above, there were accumulation of subepithelia matrix glycoproteins, goblet cell hyperplasia, and mucus over secretion and occlusion, and increased airway edema in OVA model, S-salbutamol further worsen the inflammatory condition, which was slightly improved by combination with ipratropium (IPR). R-salbutamol showed a significant therapeutic benefit against the mucus overproduction and proliferation of mucus secreting cells Combination of Ipratropium with R-salbutamol showed better therapeutic effects than R-salbutamol along.

### Example 5

### 5.1. (R)-Salbutamol Significantly Inhibited LPS-Induced M1 Macrophage Polarization, (R)-Salbutamol Downregulated Expression of Typical M1 Macrophage Cytokines

### Methods

Macrophages were treated without or with (R)/(S)-salbutamol for 1 h prior to LPS stimulation and subsequently incubated for 12 h. (R)-Salbutamol (>99% purity, 99.85% ee) and (S)-salbutamol (>99% purity, 92.73% ee) .The intracellular ROS levels were examined using a ROS indicator, DCFH-DA (Life Technologies-Thermo Fisher Scientific, MA, USA). The fluorescence of the oxidized product (2′,7′-dichlorofluorescein, DCF) was assessed using an LSM710 Laser Scanning Confocal Microscope (Carl Zeiss, Jena, Germany). The concentration of NO in the culture supernatant was measured by assaying the concentrations of nitrite (a stable NO breakdown product) using the Griess assay (Beyotime, Shanghai, China), according to the manufacturer's instructions. The intracellular NO level was detected using the NO-sensitive fluorescence probe DAF-FM DA (Sigma, St. Louis, Mo., USA) (24). Cells were labeled with DAF-FM DA (10 μM) at 37° C. for 30 min and were gently washed three times with PBS. Fluorescence was detected using a LSM710 Laser Scanning Confocal Microscope (Scale bars, 100 μm) (Carl Zeiss, Jena, Germany). Seahorse analysis: the oxygen consumption rate (OCR) and extracellular acidification rate (ECAR) of the RAW264.7 cells with or without (R)-salbutamol and induced with LPS were measured in real-time using a Seahorse XF96 extracellular flux analyzer (Agilent, Santa Clara, Calif., USA).

### Results

### (R)-Salbutamol Inhibited the Polarization of M1 Macrophages in LPS-Induced RAW264.7 Cells

To determine the cytotoxic effect of (R)-salbutamol on RAW2647 cells, cell viability was examined using a CCK-8 assay. Our data showed no changes in cell viability even when the concentration of (R)-salbutamol reached 100 μM with or without LPS (100 ng/mL). The NO and ROS generated after pretreatment with (R)-salbutamol at various concentrations (0.25 μM, 0.5 μM, 1 μM, 2 μM, 5 μM, 10 μM, and 100 μM) were examined in LPS-induced RAW264.7 cells. The concentration of (R)-salbutamol at 10 μM was selected in this study.

To investigate the effect of β2 adrenergic receptor activation on macrophage polarization, macrophages were pretreated with (R)-salbutamol and subsequently induced with 100 ng/mL LPS. Macrophages M1 (F4/80+/CD11c+/CD206−) and M2 (F4/80+/CD11c−/CD206+) polarization were analyzed using flow cytometry. Flow cytometry analysis showed that the basal level of macrophages was 24.7%, and upon stimulation with LPS, 59.2% of macrophages polarized to the M1 phenotype, suggesting that LPS could induce M1 polarization. When the macrophages were pretreated with (R)-salbutamol prior to stimulation with LPS, the prevalence of M1 macrophages decreased to 31.8%, suggesting that (R)-salbutamol alleviated the LPS-induced polarization of M1 macrophages.

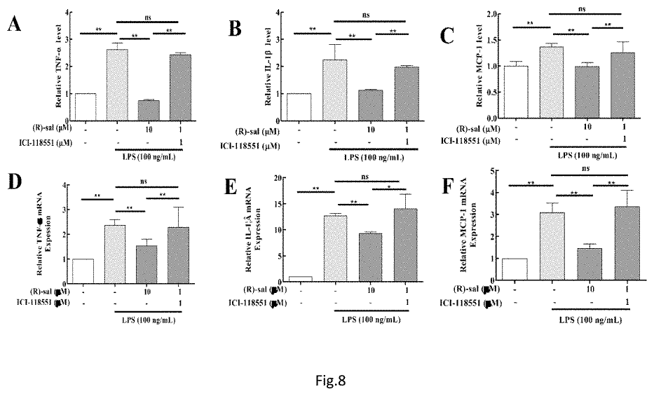

The ratio of M1 cells to the total number of cells was similar among the different treatment groups (FIG. 7), To determine the effect of (R)-salbutamol on M2 macrophage polarization, flow cytometry was used, and the data showed a decrease in the basal level of M2 macrophages from 0.244% to 0.239% and 0.214% upon stimulation with LPS and pretreatment with (R)-salbutamol, respectively. These results suggest that (R)-salbutamol has no effect on M2 polarization. Additionally, to investigate whether (R)-salbutamol mediates its effects on M1 macrophage polarization via the β2 adrenergic receptor, a specific β2 adrenergic receptor antagonist, ICI-118551, was employed in this study. This invention disclosed that the percentage of M1 macrophages increased to 55.8% when cells were pretreated with (R)-salbutamol following incubation with ICI-118551 (FIG. 7). This suggested that (R)-salbutamol exerted its inhibitory effects on M1 macrophage polarization through the β2 adrenergic receptor.

### (R)-Salbutamol Decreases the Production of TNF-α, IL-1β and MCP-1 in LPS-Induced Macrophage RAW264.7 Cells

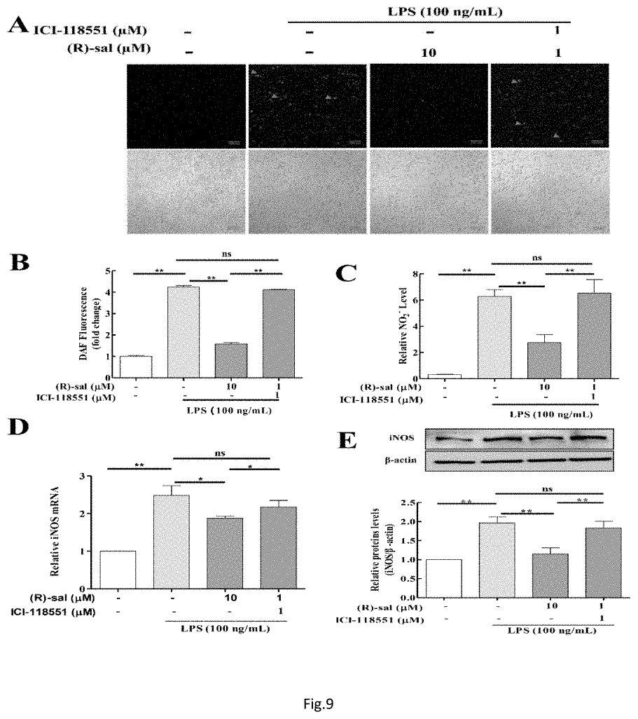

To confirm that M1 polarization was predominant over M2 polarization after stimulation with LPS, the levels of typical M1 macrophage cytokines (i.e., TNF-α, IL-1β and MCP-1) were quantified using ELISA; these cytokines are mainly synthesized by macrophages and play an important role in inflammatory diseases (35,36). This invention disclosed that TNF-α decreased from 2.62-fold to 0.75-fold of control levels following LPS stimulation (FIG. 8A). IL-1β is a hallmark of many chronic inflammatory diseases in humans and is reportedly associated with acute phase reactions (35,37). ELISA was used to quantify IL-1β, and our data showed that IL-1β was reduced from 2.24-fold to 1.12-fold of control levels following LPS stimulation (FIG. 8B). Additionally, previous studies showed that the inhibition of MCP-1 causes capillary-alveolar barrier damage and reduces the recruitment of macrophages (38-40). In this study, the concentration of MCP-1 decreased from 1.588 ng/mL to 1.15 ng/mL following LPS induction (FIG. 8C). When cells were pretreated with (R)-salbutamol prior to the stimulation of LPS, the levels of TNF-α, IL-1β and MCP-1 decreased significantly (FIG. 8A-C). We conclude that LPS is likely to lead to M1 polarization but not M2 polarization.

To examine the mRNA expression of TNF-α, IL-1β and MCP-1, quantitative real-time PCR was performed. Consistent with the cytokine expression study, the mRNA expression of TNF-α was increased by 2.26-fold in the LPS-induced group compared to the control group (FIG. 8D). The mRNA expression of IL-1β and MCP-1 increased by 12.74-fold (FIG. 8E) and 3.1-fold, respectively, in the LPS-induced group compared to the control group (FIG. 8F). When cells were pretreated with (R)-salbutamol prior to LPS stimulation, the mRNA levels of TNF-α, IL-1β and MCP-1 were significantly decreased. The mRNA expression of TNF-α, IL-1β and MCP-1 increased by 2.28-, 12.41-, and 3.36-fold, respectively, in ICI-118551-treated cells compared to the control group. Compared to LPS-induced RAW264.7 cells, the mRNA expression of TNF-α, IL-1β and MCP-1 showed no obvious changes in ICI-118551-treated cells (FIG. 8D-F), suggesting that (R)-salbutamol acts on the β2 adrenergic receptor and reduces the expression of these cytokines. These disclosure suggested that (R)-salbutamol inhibited the expression of TNF-α, IL-1β and MCP-1 via the β2 adrenergic receptor at the transcriptional level, which in turn reduced the protein expression of TNF-α, IL-1β and MCP-1 in LPS-induced macrophages.

### 5.2. Effects of (R)-Salbutamol and (S)-Salbutamol on the Production of NO and ROS in Macrophages RAW 264.7 Cells

(R)-salbutamol inhibited inducible nitric oxide synthase (iNOS) and significantly decreased the production of nitric oxide (NO) and reactive oxygen species (ROS); in contrast, (S)-salbutamol increased the production of NO and ROS.

(R)-salbutamol decreased the production of NO and ROS in LPS-induced RAW264.7 cells: LPS caused chronic inflammation usually associated with the increased production of NO. To determine the anti-inflammatory effect of (R)-salbutamol on the polarization of M1 macrophages, the intracellular NO level was determined using a NO-sensitive fluorescence probe, DAF-FM DA. Representative images revealed that the number of cells stained with DAF was increased in LPS-induced cells compared with control cells, whereas the cells pretreated with (R)-salbutamol exhibited a decreased number of stained cells compared with control cells (FIG. 9A). The level of DAF fluorescence increased by 424% in the LPS-induced group compared to the control group, while the level of DAF fluorescence decreased by 2.68-fold when cells were pretreated with (R)-salbutamol (FIG. 9B). Treatment with ICI-118551 increased NO levels, suggesting that (R)-salbutamol inhibited NO production via a β2 adrenergic receptor mechanism. In addition, the concentration of NO in the culture supernatant was measured by assaying the concentrations of nitrite (a stable NO breakdown product) using the Griess assay. Compared to control conditions, the expression of NO2-increased by 20.25-fold with LPS stimulation, and NO2-expression decreased by 2.28-fold with (R)-salbutamol pretreatment (FIG. 9C). M1 macrophages have been shown to activate iNOS to produce NO from L-arginine. To investigate (R)-salbutamol role on the level of iNOS, the mRNA and protein levels of iNOS were determined. The invention disclosed that in macrophages, LPS treatments increased the iNOS mRNA level, but it decreased when pretreated with (R)-salbutamol plus LPS (FIG. 9D). Consistent with the mRNA, the iNOS protein levels were showed same changes after treatment with LPS and LPS+ (R)-salbutamol (FIG. 9E). The effects of (R)-salbutamol blocked by adding ICI-118551.

LPS can promote cell apoptosis through mitochondrial dysfunction, and mitochondria are the major source of ROS. In this study, ROS were visualized with the DCFH-DA dye, The number of stained cells decreased in the (R)-salbutamol pretreatment group compared with the group without (R)-salbutamol pretreatment (FIG. 9A). The level of DCF increased by 7.30-fold in LPS-induced RAW264.7 cells and decreased by 3.38-fold in cells pretreated with (R)-salbutamol (FIG. 9B). This invention disclosed a opposite effects on levels of NO and ROS in LPS-induced cells, which increased by pretreating with (S)-salbutamol, and decreased by pretreated with (R)-salbutamol, Additionally, this invention disclosed we found that β2 adrenergic receptor activation is required for M1 polarization since its effects were diminished by ICI-118551, a specific β2 blocker.

This invention disclosed that the (S)-enantiomer salbutamol has different mechanisms than its (R)-enantiomer in terms of the activation of macrophages in the inflammatory response.

### 5.3. (R)-Salbutamol Increases the Ratio of GSH/GSSG in LPS-Induced Macrophages RAW264.7 Cells

The ratio of GSH to GSSG is a marker of oxidative stress. The ratio decreased to 70.60% from control levels in LPS treated cells, it increased by 84.50% when pretreated with (R)-salbutamol which was blocked by ICI-118551, the β2 adrenergic receptor blocker.

### 5.4. (R)-Salbutamol Rescued Mitochondrial Respiration and Inhibits Aerobic Glycolysis in the LPS-Induced RAW264.7 Cells

Macrophage activation elicited different metabolic profiles. LPS-induced macrophages adopted glycolytic metabolic profiles. This invention disclosed a β2 adrenergic receptor mediated Warburg metabolism (aerobic glycolysis) of in LPS treated macrophages. The OCR and ECAR were measured using an extracellular flux analyzer. LPS induced a 59.29% decrease in the OCR, a measurement of oxygen consumption rate, and (R)-salbutamol induced a 69.59% increase in the OCR in LPS-treated cells. ECAR is a measurement extracellular acidification rate, an indication of cellular glycolytic rate. LPS treatment significantly upregulated ECAR, aerobic glycolysis, compared to control conditions, while (R)-salbutamol inhibited the aerobic glycolysis. This invention disclosed that (R)-salbutamol likely mediated the metabolism shift in LPS-induced cells and that it protected against LPS-induced inflammation. This was also blocked by ICI-118551 treatment. The ratio of OCR/ECAR was reduced in LPS-treated cells, whereas the ratio of OCR/ECAR in LPS-treated cells, was increased when the pretreated with (R)-salbutamol.

### Example 6

Preventive Effects of R-Albuterol and R-Terbutaline against Respiratory Failure and Death Induced by Sepsis

Methods: Survival study: BALB/c mice were randomly assigned into different treatment groups (n=11) including: saline control (0.1 mL/10 g), (R)-salbutamol of 0.1, 0.25 or 0.5 mg/kg doses, (S)-salbutamol (0.5 mg/kg) and Dex (dexamethasone) (5 mg/kg). Treatment were given intraperitoneally for 3 consecutive days. One hour after the 3rd day treatments, LPS (15 mg/kg) was administered intraperitoneally. The animals were monitored. Survival rate was then recorded every 12 h for 144 hours.

### Lung Function Measurements

The lung functions were measured with using Buxco system (Buxco Electronics, USA). After a brief acclimation to the chamber, the mice received an initial baseline challenge of saline. Measures were made at 6 hours after administration of LPS, including tidal volume and Penh which is an indication of airway resistance.

Results: LPS administration results in death of most animals within 48 hours and rate of mortality was 90.9%. However, all the animals pretreated with high dose of R-salbutamol were survived from PLS challenge. Animals pretreated with Dexamethasone (5 mg) had the highest rate of mortality after PLS challenge. Animals pretreated with S-salbutamol had same rate of death at 48 hours, but higher rate of death at 36 h after LPS challenge in comparison of LPS challenged group without pretreatment. (table 6.1)

In addition, all the animals were survived within 6 hours after PLS challenge. The lung functions were then measured and compared between groups. LPS induced an significant suppression of respiratory function as indicated by an increased airway resistance and decreased tidal volume. This deterioration of respiratory function induced by LPS were restored in R-salbutamol pretreated animals. (table 6.2).

TABLE 6.1
Percent of death after LPS challenges within 144 hours

| Group of Treatment | | | | | | | |
|---|---|---|---|---|---|---|---|
| (mg/kg) | (n) | 12 h | 24 h | 36 h | 48 h | 72 h | 144 h |
| Saline + LPS | 11 | 0 | 36.4 | 72.7 | 90.9 | 90.9 | 90.9 |
| R-salbutamol (0.1) + | 10 | 0 | 20 | 20 | 40 | 40 | 40 |
| LPS | | | | | | | |
| R-salbutamol (0.5) + | 11 | 0 | 0 | 0 | 0 | 0 | 0 |
| LPS | | | | | | | |
| S-salbutamol (0.5) + | 11 | 0 | 36.4 | 90.9 | 90.9 | 90.9 | 90.9 |
| LPS | | | | | | | |
| R-terbutaline (0.5) + | 11 | 0 | 18.2 | 27.3 | 36.4 | 36.4 | 36.4 |
| LPS | | | | | | | |
| Dexamethasone (5.0) + | 11 | 0 | 90.9 | 90.9 | 100 | 100 | 100 |
| LPS | | | | | | | |
| R-salbutamol (0.5) + | 11 | 0 | 0 | 0 | 0 | 0 | 0 |
| saline | | | | | | | |

TABLE 6.2
Reparatory functions at 6 hours after LPS challenges

| | | Index of lung function | |
|---|---|---|---|
| Group (mg/kg) | (n) | Penh | Tidal volume (mL) |
| Saline | 10 | 0.54 ± 0.09* | 0.22 ± 0.02* |
| LPS + saline | 11 | 1.31 ± 0.40 | 0.13 ± 0.03 |
| LPS + R-sal (0.1) | 10 | 0.68 ± 0.09* | 0.17 ± 0.02* |
| LPS + R-sal (0.5) | 10 | 0.53 ± 0.07* | 0.21 ± 0.02* |
| LPS + Dex (5.0) | 10 | 1.01 ± 0.46 | 0.15 ± 0.04 |

*p < 0.05, vs LPS; Dex (Dexamethasone)

1. A method of use sufficient optic pure R-enantiomer β2 agonists and its pharmaceutical acceptable salts as immune-modulators in manufacture of pharmaceutical medicaments for prevent or treatment of acute or chronic bronchia-lung inflammatory symptoms or inflammatory remolding while avoiding immune-inflammation related adverse effects.

2. The method of claim 1, wherein the said R-enantiomer β2 agonists have enantiomer excess value of 50%-85%.

3. The method of claim 1, wherein the said R-enantiomer β2 agonists have enantiomer excess value of 85%-98%.

4. The method of claim 1, wherein the said R-enantiomer β2 agonists have enantiomer excess value of 98%-99.9%.

5. The method of claim 1, wherein the R-enantiomer β2 agonists is terbutaline, salbutamol, bambuterol, vilanterol, clenterol, salmeterol, formoterol, trantinterol and Indacaterol

6. The method of claim 1, wherein the said R-enantiomer β2 agonists are R or R′ R′ enantiomers of Short-acting β2 agonists: bitolterol, fenoterol, isoprenaline, orciprenaline ormetaproterenol, pirbuterol, procaterol, ritodrine; and Long-acting β2 agonists: bambuterol, arformoterol, Formoterol, Perforomist; and Ultra-long-acting β2 agonists: abediterol, carmoterol, indacaterol, Olodaterol, vilanterol, isoxsuprine, mabuterol and zilpaterol.

7. The method of claim 1, wherein, the said inflammatory symptoms are pneumonia or bronchitis, acute respiratory distress syndrome in response to bacteria, virus or other pathogen-associated molecular patterns (PAMP).

8. The method of claim 1, wherein, the said chronic inflammatory symptoms are emphysema or persistent chronic bronchitis with pneumonic exacerbations

9. The method of claim 1, wherein, the said inflammatory symptoms are characterized by activation or proliferation of M1 type macrophages or/with enhanced aerobic glycolysis in lungs or blood.

10. The method of claim 1, wherein, the said inflammatory symptoms are characterized by increased eosinophils in lungs and blood.

11. The method of claim 1, wherein, the said inflammatory symptoms are characterized by increased monocyte chemoattractant protein-1(MCP-1) in lungs or blood.

12. The method of claim 1, wherein, the said inflammatory symptoms are characterized by increased cytokines including, TNF-α, IL-1β, IL-2, IL-4, IL-5, IL-6 and IL-13 in lungs or blood.

13. The method of claim 1, wherein, the said inflammatory symptoms are characterized by increasing in the ratio of phosphorylated p38 MAPK to total p38 MAPK and the ratio of phosphorylated p65 NF-κB to total p65 NF-κB.

14. The method of claim 1, wherein, the said inflammatory symptoms are characterized by increasing in serum IgG.

15. The method of claim 1, wherein, the said inflammatory symptoms are characterized by increasing in ROS and ON productions in lung and blood or in expression of inducible nitric oxide synthase (iNOS).

16. The method of claim 1, wherein, the said inflammatory remolding are emphysema, mucus hypersecretion, inflammatory sputum production and airway fibrosis.

17. The method of claim 1, wherein, the said inflammatory remolding is characterized by sub-epithelial fibrosis, myofibroblast hyperplasia, airway and vascular smooth muscle hypertrophy, increased thickness of bronchiole wall and vascular vessels, mucus gland and goblet cell hyperplasia, and epithelial disruption and excessive interstitial deposition of collagen and increased alveolar septum or gas/blood barrier.

18. The method of claim 1, wherein, the said inflammatory remolding is characterized by increased pulmonary arteriole resistance, pulmonary arteriole or vascular remodeling and pulmonary hypertension.

19. The method of claim 1, wherein, the said adverse effects are frequent use of S-enantiomer or (RS)- racemic beta2 agonists related adverse effects.

20. The method of claim 1, wherein, the said adverse effect is characterized by airway hyper-responsiveness to allergy-genic agents, due to frequent use of (S)-enantiomer or (RS)-racemic beta 2 agonists.

21. The method of claim 18, wherein, the said S-enantiomer related adverse effects are characterized by further enhancing the proliferation and remodeling of bronchiole smooth muscle and exacerbation in asthma-COPD conditions.

22. The method of claim 19, wherein, the said S-enantiomer related adverse effects are characterized by further enhancing the proliferation of fibroblasts and interstitial collagen deposition and increase in alveolar septum and gas/blood barrier, reduced blood oxygen in asthma-COPD or respiratory failure conditions with deterioration of lung functions as results of frequent use of RS-racemic β2 agonists,

23. The method of claim 1, wherein, the said adverse effects related to use racemic terbutaline or racemic salbutamol for tocolytic therapies or preterm labor.

24. The method of claim 1, wherein, the pharmaceutical medicaments are solid dose forms, gel, suppository, liquid or lyophilizes powder for injections, ointment or patches for topic use and aerosol or dry powders for lung inhalation or nasal.

25. The method of claim 1, wherein, the said treatments are characterized by administration of the said medicaments by inhalation via lung or nasal.

26. The method of claim 1, wherein, the said treatments are characterized by administration of the said medicaments by oral, by injection intravenously or subcutaneously and as suppository in rectum or vagina.

27. The method of claim 1, wherein, the said treatment is characterized by administration of the said medicaments in combination with ipratropium or other muscarinic receptor blockers.

28. The method of claim 1, wherein, the said treatment is characterized by administration of the said medicaments in combination with ipratropium or other muscarinic receptor blockers and corticosteroids.

29. The method of claim 27, wherein, the said muscarinic receptor blockers are tiotropium, glycopyrronium, aclidinium and umeclidiniumt.

30. The methods of claim 28, wherein, the said corticosteroids are budesonide, fluticasone, ciclesonide, beclomethasone, mometasone, flunisolide, prednisolone and triamcinolone acetonide.

31. The method of claim 1, wherein, the said pharmaceutical acceptable salts are hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.
